(19) United States
(12) Patent Application Publication
(10) Pub. No.: US 2001/0049117 A1
(43) Pub. Date: Dec. 6, 2001

(54) ANALOGS OF UDP-MURNAC PEPTIDES, ASSAYS, KITS AND RELATED METHODS OF THEIR USE

(76) Inventors: HELENA R. AXELROD, WEST WINDSOR, NJ (US); ARTHUR A. BRANSTROM, HIGHTSTOWN, NJ (US)

Correspondence Address: ADVANCED MEDICINE, INC., 901 GATEWAY BOULEVARD, SOUTH SAN FRANCISCO, CA, 94080, US

(21) Appl. No.: 09/375,505

(22) Filed: Aug. 17, 1999

Related U.S. Application Data
(60) Provisional application No. 60/097,324, filed on Aug. 20, 1998.

(51) Int. Cl. G01N 33/554; G01N 33/569

(52) U.S. Cl. 435/7.32

(57) ABSTRACT

General compositions and methods for detecting Lipid I, Lipid II and peptidoglycan synthesis is disclosed. A method of screening for potential antibacterial agents is provided which requires bacterial membrane preparations or enriched enzyme preparations including at least one bacterial enzyme involved in the synthesis of Lipid I from UDP-MurNAc pentapeptide and undecaprenyl phosphate, at least one bacterial enzyme involved in the synthesis of Lipid II from Lipid I and UDP-GlcNAc and one or more bacterial enzymes involved in the further processing of Lipid II toward the downstream synthesis of peptidoglycan. The methods disclosed herein further provides a labeled UDP-MurNAc-peptide capable of serving as a substrate for a bacterial enzyme involved in the synthesis of Lipid I and a labeled UDP-GlcNAc capable of serving as a substrate for a bacterial enzyme involved in the synthesis of Lipid II. Conditions for further processing of Lipid II toward the downstream synthesis of peptidoglycan are also discribed.

### 1. FIELD OF THE INVENTION

The present invention relates to analogs of the certain substrates of bacterial cell wall biosynthesis enzymes. The action of the enzymes of interest on the analogs, in the presence of components found in the bacterial membrane or provided exogenously provide for a variety of products of bacterial cell wall biosynthesis, including Lipid I, Lipid II, peptidoglycan, and their labeled and unlabeled analogs. The formation of peptidoglycan precursors, Lipid I and Lipid II, is thought to be catalyzed by the expression products of the MraY and MurG genes. These enzymes are believed to be involved in the first and second steps of the lipid cycle reactions, respectively. The analogs and compositions of the invention find particular utility in rapid high throughput assays for identifying compounds that inhibit one or more steps involved in the biosynthesis of bacterial cell wall. In particular, an analog of uridine diphosphate-N-acetylmuramyl peptide or analog of “UMP” is disclosed that serves as a substrate for the enzyme that catalyzes the production of Lipid I from UMP and undecaprenyl phosphate. Methods are also disclosed for assessing the activities of the putative enzymes MraY and MurG in tandem, in particular sample preparations with or without the presence of a suspected enzyme inhibitor, e.g., in an assay for the identification of potential antibacterial drug candidates.

### 2. BACKGROUND OF THE INVENTION

2.1. Biosynthesis of Peptidoglycan

Biosnthesis of peptidoglycan in bacteria is a complex process involving numerous enzymes, most of which have been shown to be essential in pathogenic bacteria. These enzymes have been studied most extensively in E. coli, and the genes that encode them have been cloned. The protein expression products have been charcaterized over the past several years. (See, e.g., Pucci et al., in J. Bacteriology (1997) 179:5632-5635.) The biosynthesis of gram-positive bacteria is as well understood. However, recently it has been found that there are similarities in the genetic background and the mode of action of several of the of the cell membrane associated enzymes of gram-positive and gram-negative bacteria. (Ssee, e.g., (a) K. V. I. Rolston, et al., in Journal of Antimicrobial Chemotherapy (1996) 38:265-269; (b) David Landman, et al., in Journal of Antimicrobial Chemotherapy (1996) 37:323-329; (c) E. A. Somner et al., in Antimicrobial Agents and Chemotherapy (1990) 34:413-419; (d)Masato Ikeda, et al., in J. Gen. Appl. Microbiol. (1990)36:179-187; (e) Philip E. Bransish, et al., in Antimicrobial Agents and Chemotherapy (1996) 40:1640-1644; (f) Michael J. Pucci, et al., in Journal of Bacteriology (1997) 179:5632-5635; (g) Francis C. Neuhaus, in Accounts of Chemical Research (1971) 4:297; (h) N. H. Georgopapadaukou, in Antimicrobial Agents and Chemotherapy (1993) 37:2045-2053; (i) Labischinski et al., in (Ghuysen and Hackenheck (eds) Bacterial Cell Wall. (1994) 23; (j) Buchnan et al., in (Ghuysen and Hackenheck (eds) Bacterial Cell Wall. (1994) and in Science B.V. (1994) 167.

The biosynthesis of peptidoglycan is catalyzed by a series of membrane-associated enzymes that utilize two nucleotide-activated precursors, UDP-N-acetylglucosamine or “UDP-GlcNAc” and UDP-N-acetylmuramyl pentapeptide (or more generally, UDP-N-acetylmuramyl “peptide”). (See, e.g., Ghuysen and Shockman, 1973.) Because interference with peptidoglycan biosynthesis is a proven strategy for treating bacterial infections, all of the enzymes involved in peptidoglycan biosynthesis are potential targets for the development of new antibiotics.

The emergence of resistance to existing antibiotics has rejuvenated interest in bacterial enzymology. It is hoped that detailed mechanistic and structural information about bacterial enzymes involved in critical biosynthetic pathways could lead to the development of new antibacterial agents. While some detailed structural and mechanistic information on some of the early enzymes in the pathway is now available, most of the downstream enzymes have proven very difficult to study.

Some of the best antibiotics function by interfering with the biosynthesis of the peptidoglycan polymer that surrounds bacterial cells. With the emergence of bacterial pathogens that are resistant to common antibiotics, it has become imperative to learn more about the enzymes involved in peptidoglycan biosynthesis. Although remarkable progress has been made in characterizing some of the early enzymes in the biosynthetic pathway (see, e.g., (a) Fan, C. et al., in J. R. Science (1994) 266:439; (b) Benson, T. E. et al., in J. M. Nat. Struct. Biol. (1995) 2:644; (c) Jin, H. Y. et al., in J. J. Biochemistry (1996) 35:1423; (d) Skarzynski, T. et al., in Structure (1996) 4:1465; (e) Schonbrunn, E. et al., in Structure (1996) 4:1065; (f) Benson, T. E. et al., in Biochemistry (1997) 36:806), little progress has been made with the study of the downstream enzymes.

Part of the difficulty stems from the fact that such downstream enzymes are membrane-associated. (See, e.g., (a) Gittins, J. R. et al., in Microbiol. Rev. (1994) 13:1; (b) Bupp, K. and van Heijenoort, J. (1993) 175:1841.) This fact makes them intrinsically hard to handle. A separate difficulty is that the substrates for many of these enzymes are not readily available. (See, e.g., (a) Pless, D. D. and Neuhaus, F. C., in J. Biol. Chem. (1973) 248:1568; (b) van Heijenoort, Y. et al., in J. Bacteriol. (1992) 174:3549.) These problems have impeded the development of activity assays suitable for detailed mechanistic investigations of the downstream enzymes. For a fluorescent assay to monitor MraY activity, see, e.g., Brandish, P. E. et al., in J. Biol. Chem. (1996) 271:7609.

2.2. MraY

One such downstream enzyme involved in peptidoglycan biosynthesis is MraY. MraY catalyzes one of the final intracellular steps in the biosynthetic pathway of bacterial cell wall or peptidoglycan biosynthesis, i.e., the transfer of UDP-N-acetylmuramyl-pentapeptide to undecaprenyl phosphate to provide Lipid I. Lipid I, in turn, is the substrate for murG. This process involves the enzymatic transfer of cytoplasmic precursors to the membrane where intermediates covalently linked to undecaprenyl phosphate are assembled and translocated across the membrane to the site of nascent peptidoglycan synthesis.

Phospho-N-acetylmuramyl-pentapeptide-translocase (also translocase 1) catalyzes the transfer of phospho-N-aceylmuramyl-L-Ala-gama-D-Glu-m-DAP-D-Ala-D-Ala from uridine 5′ monophosphate to a membrane-bound lipid carrier, undecaprenyl phosphate. This enzyme is encoded by the MraY gene in E. Coli. This gene has been cloned and sequenced. The examination of amino acid sequence of the protein indicates that the encoded enzyme is an integral membrane protein. See, Ikeda et al., in J. Bacteriol. (1991) 173:1021.

However, up to the present time, neither the mechanism of intramembrane translocation nor the membrane environment surrounding the lipid intermediates has been established. Utilization of labeled substrate, UDP-MurNAc pentapeptide, provides a specific mecahnism for evaluating the micro environments within the membrane which are formed during the biosynthetic sequence. For general information on biosynthesis of peptidoglycan, see, e.g., William Weppner et al., in The Journal of Biological Chemistry (1978) 253:472.

The MraY substrate can be readily isolated in large quantities from bacterial cultures. Typically, the membrane preparations can be supplemented with exogenous UDP-N-acetyl muramic acid pentapeptide for conversion to Lipid I. The lipid-linked Lipid I product of MraY is further elaborated by attachment of a N-acetylglucosamine unit, and the precursor is somehow flipped across the membrane and incorporated into peptidoglycan. This and other such lipid-linked cycles have been reviewed. See, e.g., Bugg et al., in Microbiol Letter (1994) 119:255.

It has been reported that no commercial antibiotics in current use are directed against Lipid I, thus enzymes involved in the formation of Lipid I provide novel targets for new antibacterial agents. See, e.g., Brandish et al., in The Journal of Biological Chemistry (1996) 271:7609. Until recently, the only known inhibitor of this step of peptidoglycan biosynthesis was tunicamycin, which is known also to inhibit mammalian glycoprotein biosynthesis and other lipid-linked glycosyl transfer reactions, and amphomycin, which chelates undecaprenyl phosphate in the presence of calcium. Ssee, e.g., Tamura et al., in Biochem. Biophys. Research Commun. (1976) 40:447; Banerjee, et al., in Journal of Biol. Chem. (1989) 264:2024.

There is a demonstrated need in the art for a direct assay that can be used for effective screening of novel, carbohydrate based libraries for potential anti-infective drugs. An unmet challenge for developing assays has been to design a system that will be specific for the targeted enzymes and be amenable to high throughput testing. The inventors of the present invention have developed such an assay, which is based upon the discovery of an anolog of the Lipid I precursor, UDP-MurNAc pentapeptide, and the use of a labeled UDP-GlcNAc substrate, both of which would be incorporated into a Lipid II analog.

The assay disclosed and described herein allows for the separation and identification of lipid products produced by the enzymatic activity of inter alia the MraY and MurG proteins. This assay system uses bacterial membrane preparations, e.g., those obtained from E. coli. cells overexpressing the MraY and MurG gene products, as a source of enzymes. The results disclosed and described herein reveal that at least 20-25% of the detectable label associated with GlcNAc is converted into lipid-linked products, while no incorporation is observed in reactions lacking UDP-MurNAc pentapeptide.

Among the advantages of the present assay is its ability to measure the activity of more than one enzyme at a time. Moreover, it is anticipated that the assay can be applied to both gram negative and gram positive bacteria. The assay has the ability to detect inhibitors specifically affecting the gene products of MraY and/or MurG, but is flexible enough to uncover inhibitors of other downstream enzymes. Further, its simplicity lends itself well to high throughput screening.

### 3. SUMMARY OF THE INVENTION

An analog, composition, assay kit and methods for the detection bacterial cell wall biosynthtic enzyme acitivity are disclosed. For the first time a method is disclosed that has the ability to measure the activities of the cell membrane associated enzymes in tandem. In particular, what is disclosed is an analog of uridine diphosphate N-acetylmuramyl peptide of the formula (UMP)-X, wherein the group UMP represents a uridine diphosphate N-acetylmuramyl peptide and the goup X represents a capture moiety that is directly or indirectly attached to said UMP and which capture moiety permits the separation of any substance to which the capture moiety is attached from a mixture comprising such substance. Consequently, the substance to which the capture moiety is attached can isolated.

Also disclosed herein is a composition for the detection of the products or precursors of peptidoglycan biosynthesis in bacteria, which comprises a labeled uridine diphosphate N-acetylmuramyl peptide, preferably a pentapeptide (UDP-MurNAc pentapeptide) and a labeled uridine diphosphate N-acetylglucosamine (UDP-GlcNAc).

Consistent with the objectives of this invention, an analog is disclosed for the detection of the formation of Lipid I, Lipid II, or peptidoglycan comprising an isolated uridine diphosphate N-acetylmuramyl-peptide (UDP-MurNAc-peptide) attached to a capture moiety, provided that the capture moiety. Preferably, the analog of the invention does not bear a radioactive moiety, a fluorescent moiety, or a metal moiety.

In a preferred embodiment of the invention, the group X is attached to lysine or meso-DAP of the UDP-MurNAc peptide and the peptide is either a tripeptide, a tetrapeptide or a pentapeptide. In another preferred embodiment of the invention the group X attached to the UDP-MurNAc-peptide is a biotin moiety attached either directly or indirectly to an amino acid residue, preferably lysine via the epsilon amine.

Also disclosed herein is a method for detecting production of Lipid I, a Lipid I-like substance, or a Lipid I analog in a test sample. The label according to this invention can be attached either directly or indirectly and may be selected from the group consisting of radioactive, fluorescent, metal, enzymes, biotin, chelators, peptides, nucleic acids, receptors and lectins. Lipid II, Lipid II-like substances, Lipid II analogs and peptidoglycan moieies can also be detected according to the methods of the invention.

A method is disclosed of screening for potential antibacterial agents comprising: (a) providing a bacterial membrane preparation or enriched enzyme preparation including at least one bacterial cell wall biosynthesis enzyme involved in the synthesis of Lipid I from UMP and undecaprenyl phosphate; (b) providing an amount of an inhibitor effective to inhibit further processing of any Lipid I or Lipid I analog; (c) providing an analog of UMP capable of serving as a substrate for the at least one bacterial cell wall biosynthesis enzyme involved in the synthesis of Lipid I, to provide a Lipid I analog; (d) determining a baseline amount of Lipid I analog produced from the above-recited steps; and (e) comparing such baseline amount with a test amount of Lipid I analog produced under the same conditions used to provide such baseline amount except for the presence of an added amount of a test agent suspected of exhibiting antibacterial activity. Modifications of the above-mentioend screening method are also described, which permit the discovery of inhibitors of further downstream cell wall biosynthesis enzymes.

Consistent with the objectives of this invention an assay kit comprising (a) an analog of UMP capable of serving as a substrate for at least one bacterial cell wall biosynthesis enzyme involved in the synthesis of Lipid I; (b) a labeled UDP-GlcNAc capable of serving as a substrate for at least one bacterial cell wall biosynthesis enzyme involved in the synthesis of Lipid II; and (c) a capture agent.

The diagnostic test kit according to this invention could further comprises a bacterial membrane preparation or enriched enzyme preparation including at least one bacterial enzyme involved in the synthesis of Lipid I, Lipid II, or peptidoglycan. Inhibitors of further downstream processing of Lipid I or Lipid II can also be included. The inhibitors could be selected from known inhibitors of Mra Y and MurG enzymes including but not limited to ramoplanin, and tunicamycin.

Yet another object of this invention is to provide an assay effective to detect drugs that inhibit or reduce growth of the gram positive bacteria, comprising an enzyme, capable of specific binding to Lipid II or to an epitope of Lipid II that has adhered to a biotinylated MurNac-peptide; and a substrate for the detection of streptavidin/biotin binding. The enzyme could be, for example, alkaline phosphatase and the substrate could be, for example, 2-bromochloroindolyl phosphate nitroblue tetrazolium (BCIP/NBT.)

### 4. BRIEF DESCRIPTION OF THE FIGURES

FIG. 1. GlcNAc Transerase Assay-Time Course Paper Chromatography Results (0.1% Triton in reaction buffer). All reactions are run with 0.1% Triton in reaction buffer. The conditions for the GlcNAc transferase assay are optimized to allow for the greatest conversion of radioactive GIcNAc into Lipid II, and for a minimized formation of peptidoglycan. Paper chromatography results demonstrate that when 0.1% Triton X-100 is added to the reaction mixture, the formation of peptidoglycan is inhibited (peptidoglycan remains at the origin on a paper chromatogram, while Lipid II migrates with a relative mobility to the solvent front of 0.8-this is approximately 15 cm on the graphs). Optimal conditions are determined to be 15 minute incubations in the presence of 0.1% Triton for the buffer conditions being employed.

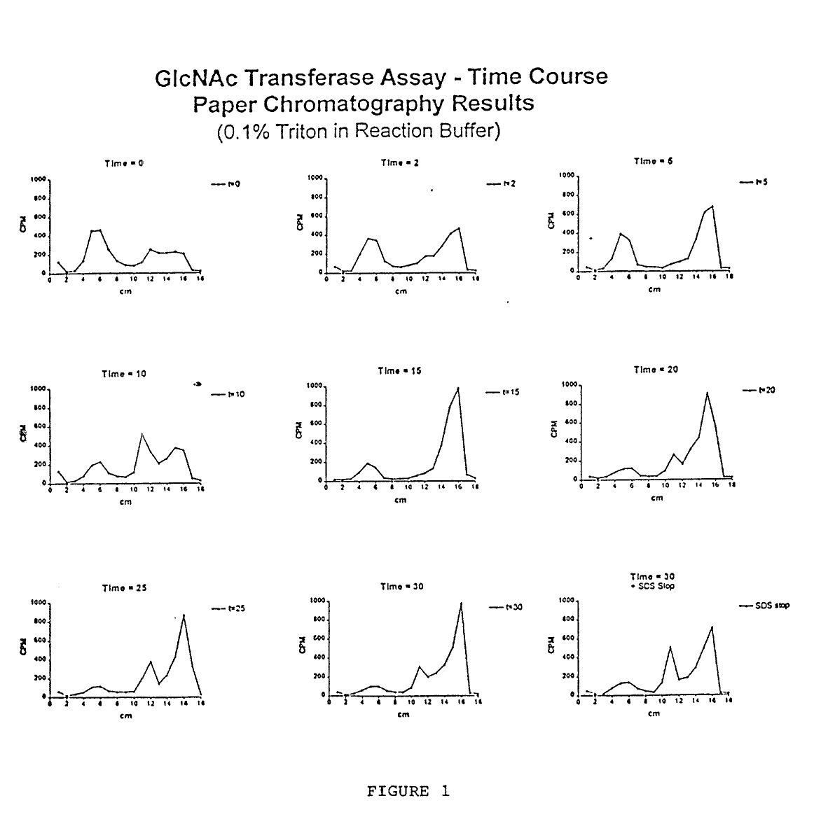

FIG. 2. Titration of Biotinylated UDP-GlcNAc. This assay demonstrates that the amount of biotinylated UDP-MurNAc pentapeptide supplied to the reaction is directly proportional to the amount of radioactive product captured by the streptavidin coated beads. The UDP-MurNAc pentapeptide is therefore a rate limiting component of the assay and is shown to be titratable with respect to radioactive GlcNAc incorporation.

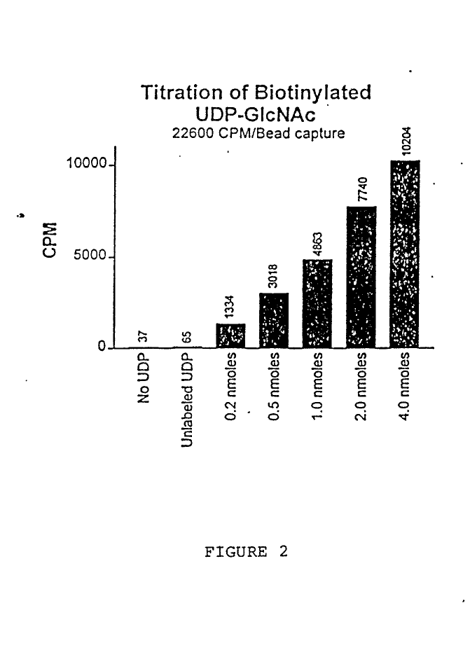

FIG. 3. GlcNAc Transferase Assay: Antibiotic Effects. The effects of suspected MraY/MurG inhibitors are identified by testing the sensitivity of the enzymatic reaction to potential antibiotics. Three known inhibitors are all greatly active, having IC₅₀ values less than 1 ug/ml in this experiment. The demonstration of high signal to noise ratios and the titration of known inhibitors underscores the potential for using this assay as a screening mechanism to identify inhibitors of MraY/MurG enzymes.

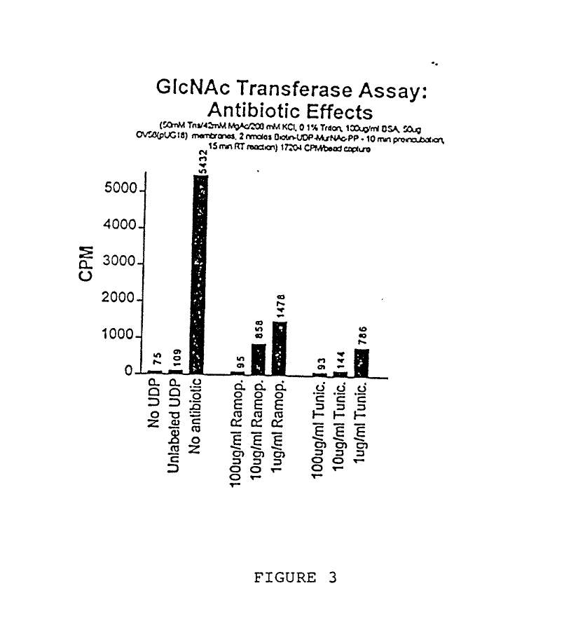

FIG. 4. GlcNAc Transferase Assay: Testing Ampicilin As An Inhibitor. The antibiotic ampicillin has no effect on the coupled GlcNAc transferase assay which measures the formation of Lipid II by the enzymes MraY and MurG. Ampicillin affects the later stages of bacterial cell wall synthesis. This supports the experimental design of the assay that an antibiotic not specifically directly against Lipid I/Lipid II formation will have no effect on the incorporation of radioactive GlcNAc with a biotinylated-MurNAc peptide-containing lipid. Similarly, the addition of 100 ug/ml moenomycin (a terminal cell wall synthesis inhibitor of transglycosylase activity) did not affect the formation of streptavidin-capturable radiolabeled Lipid II.

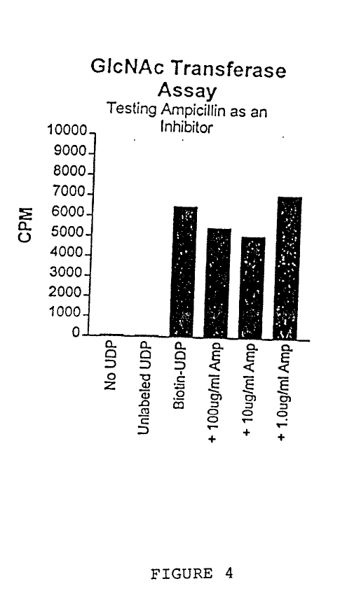

FIG. 5. GlcNAc Transferase Assay. OV58 (PUG18) vs. 23226 Membranes. The coupled assay has been optimized using E. coli OV58(pUG18) membranes which overexpress the MurG enzyme. To evaluate the versatility of the assay for other bacterial membranes, wild-type E. coli bacterial membranes are prepared from the ATCC strain #23226. While specific capture of radioactive Lipid II product is approximately 5 fold less with the 23226 membranes than with constructs overexpressing MurG, the signal to noise ratios have been demonstrated to be very high which would allow for these membranes to be utilized in future assays. In addition, the use of wild-type membranes does not foreclose the possibility of developing a coupled MraY/MurG assay to monitor potential inhibitors of gram positive bacteria.

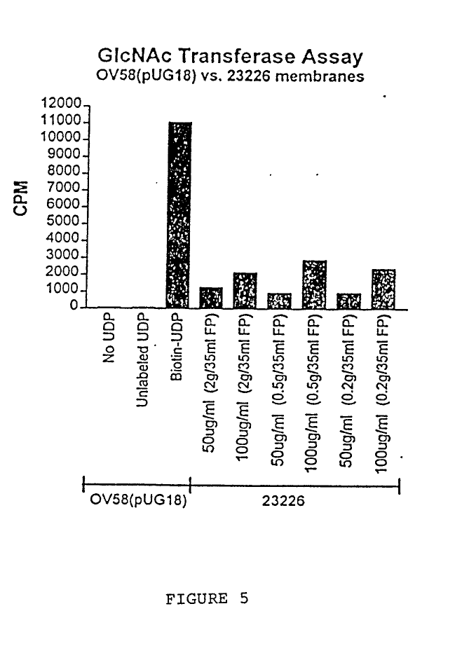

FIG. 6. GlcNAc Transferase Assay. Time course Paper Chromatography (no Triton in reaction buffer). Present assay conditions include the presence of 0.1% Triton. Preliminary results indicate that the biotinylated substrate (UDP-MurNAc pentapeptide) and radiolabeled GlcNAc would be incorporated into peptidoglycan if the reaction is allowed to proceed for a longer period of time in the absence of Triton X-100. Captured counts indicate that both Lipid II and peptidoglycan are captured by the streptavidin coated beads.

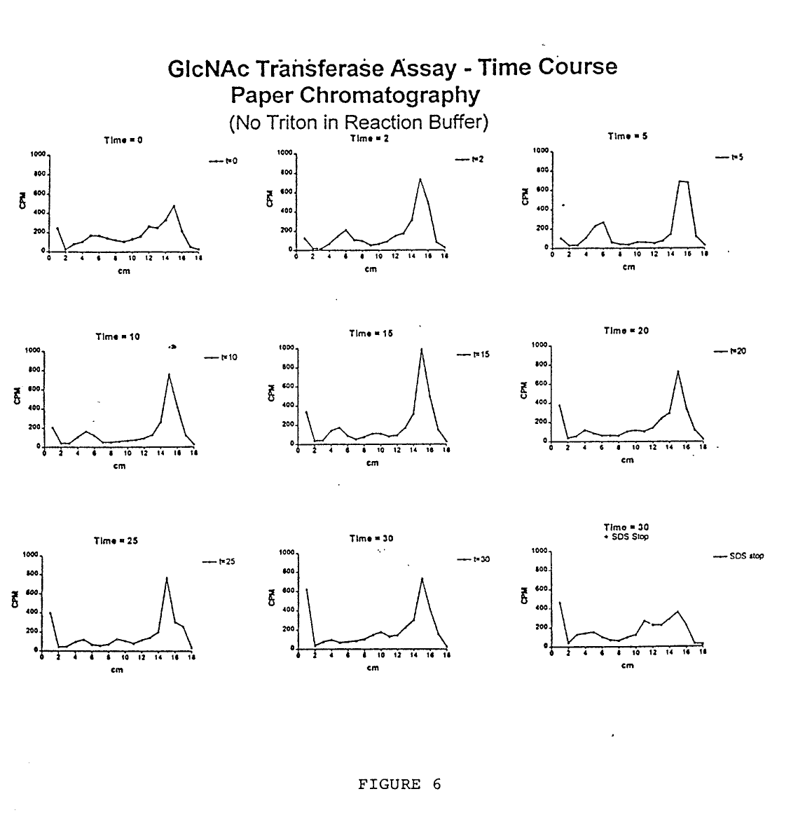

FIG. 7. Peptidoglycan Polymerization Assay: Effect of increasing ETB cell wall protein and ¹⁴C-UDPGlcNAc substrate. Ether treated bacterial protein is subjected to vancomycin in the presence of 0.5-1 micromolar of ¹⁴C-UDP-GlcNAc. Incorporation of radioactivity in the bacterial protein is measured.

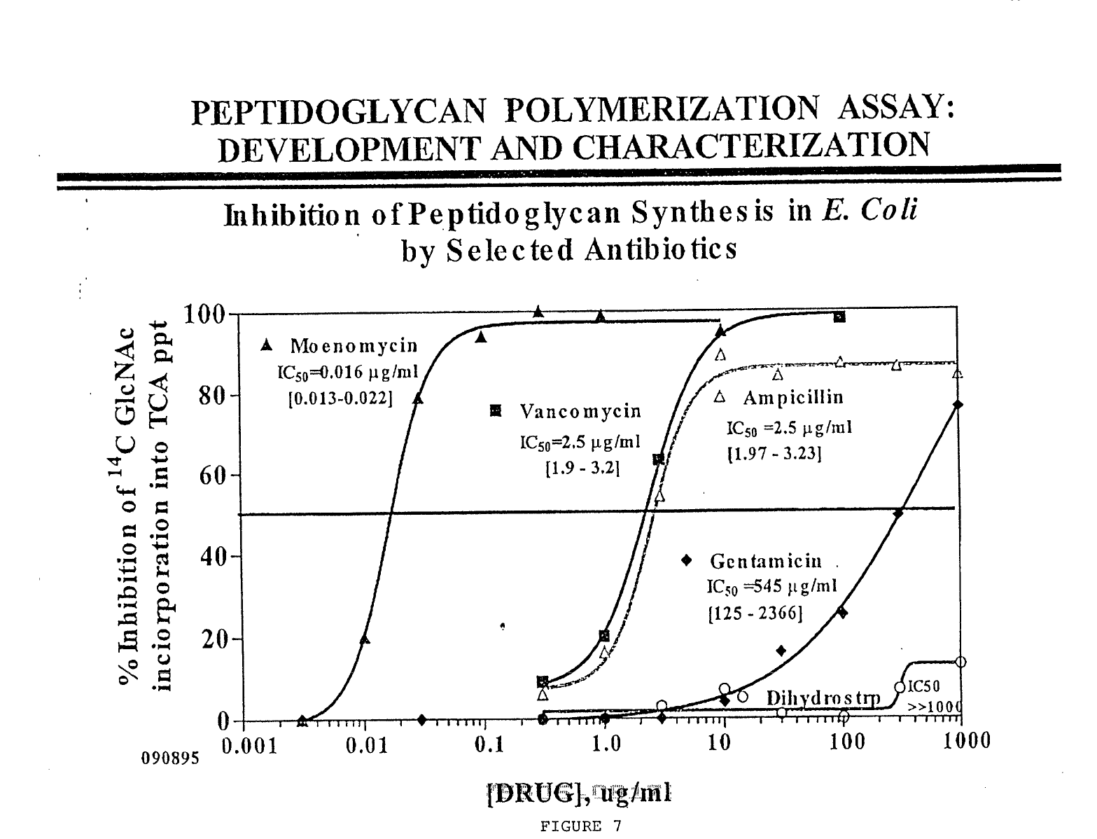

FIG. 8. Inhibition of peptidoglycan synthesis in E. Coli by selected antibiotics, such as moenomycin, vancomycin, and ampicillin.

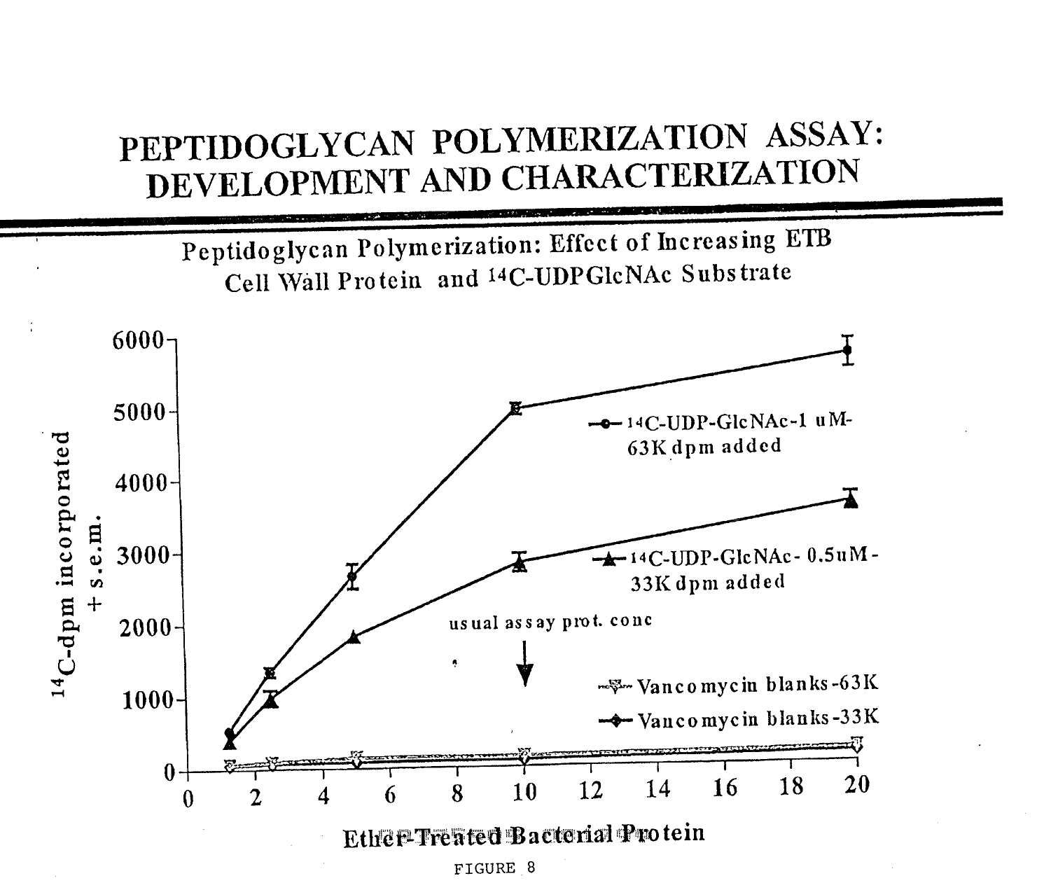

FIG. 9. Lipid II Formation Assay (Gram Positive Membrane): S. aureus and S. epidermidis membranes from gram positive organisms show an increase of ¹⁴C incorporation over background and indicates that the enzymes are titratable. This suggests that the Lipid II product of gram positive bacteria could be captured by this assay.

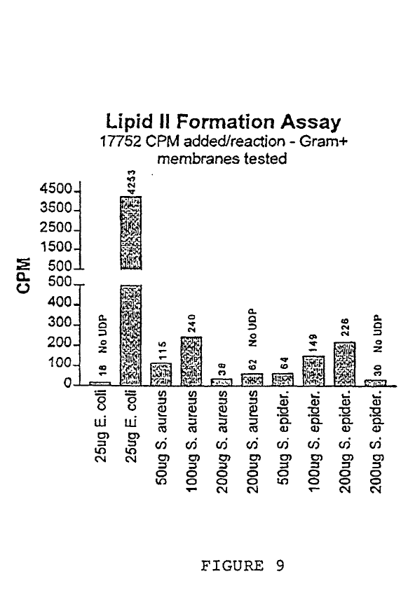

### 5. GLOSSARY

To assist the reader in gaining a better understanding of the present invention, the following terms are defined.

Substance: A matter of particular or definite chemical constitution. As disclosed herein, a substance can include small molecules, peptides, proteins, carbohydrates, nucleic acids and combinations, derivatives, homologs and analogs thereof. An analog of a particular substance includes a modified form of the substance, such as the addition, removal, or substitution of particular consituents of the initial substance. In the present invention, an analog is frequently referred to in the context of a modied UMP, Lipid I, Lipid II, or peptidoglycan.

Label: A “label” or “labeled” substance means that an original substance is modified to include or incorporate a label such that the label permits the detection, capture, or, otherwise, monitoring of the labeled substance, which labeled substance may have undergone or participated in a chemical transformation, particularly, but not limited to, those transformations that are mediated by bacterial cell wall biosynthesis enzymes of potential interest. A “label” is also useful for distinguishing a compound by introducing thereto a traceable constituent. The label can take many forms, including but not limited to conventional radioisotopic labeling; chemical labeling, including metals, chelators, peptides, nucleic acids, receptors, lectins; immunogenic labeling, or a label with light scattering effect, and the like. Suitable methods to detect such labels are scintillation counting, autoradiography, fluorescence measurement, calorimetric measurement, or light emission measurement. In certain embodiments of the invention, a label may include a capture moiety, as defined further below.

Thus, the labeling may comprise a radiolabel (e.g. ¹⁴C, ³²P, ³H, and the like), an enzyme (e.g., peroxidase, alkaline or acid phosphatase, and the like), a bacterial label, a fluorescent label, an antibody (which may be used in a double antibody system), an antigen (to be used with a labeled antibody), a small molecule such as biotin (to be used with an avidin, streptavidin, or antibiotin system), a latex particle (to be used in a buoyancy or latex agglutination system), an electron dense compound such as ferritin (to be used with electron microscopy), or a light scattering particle such as colloidal gold, or any combinations or permutations of the foregoing.

For example, if the labeling portion of a substrate probe is an antigen, a signal can be generated by complexing the antigen with an antibody/enzyme conjugate, followed by addition of an enzyme substrate. If this portion were an antibody, signal can be generated by complexing anti-antibody or an F_{C} binding protein such as Protein A therewith, when such second antibody or Protein A have been conjugated to an enzyme.

For reasons of ease and safety in the handling of the assay, it is preferred that it be chemically labeled, especially enzymatically or immunologically. In more preferred embodiments, the chemical label of choice is a hapten such as biotin, iminobiotin, fluorescein and the like.

Among the preferred labeling systems, more specifically, a capture moiety that may be mentioned are those based on the biotin/strepavidin system. This system can be incorporated into the probe by a variety of means. For example, the probe can be covalently attached to biotin via a cytochrome C bridge (see, Manning et al., in Biochemistry (1977) 16:1364-1370; Manning et al, in Chromosoma (1975) 53:107-117; Sodja, A., in Nucleic Acids Research (1978) 5:385-401)), or it can be covalently incorporated into specific nucleotide residues (see, Langer, P. R., PNAS, USA (1981) 78:6633-6637), or the biotin can be attached to a polynucleotide by means of a diamine (e.g., pentane diamine) bridge (Broker, T. R., et al, in Nucleic Acids Research (1978) 5:363-384). Interaction of the biotin molecules with avidin, streptavidinor antibiotin antibodies is then carried out, wherein the avidin, streptavidin or the other moiety of interst is conjugated to such signaling components as latex particles (see, Sodja, A., et al, supra, or Manning, et. al., in Chromosoma, supra) ferritin (see, Broker, supra) a fluorogen such as fluorescein, an enzyme, secondary antibodies, magnetic particles, or the like.

Peptidoglycan: A chemical composition which is part of the cell wall and consists of repeating subunits of crosslinked N-Acetyl glucosamine and N-acetylmuramic acid.

Capture Agent: A substance, generally bound to a solid substrate, which is capable of capturing and separating another substance that is labeled, or more specifically, which bears a capture moiety, from a mixture comprising the substance bearing the label or capture moiety. The capture could be biochemical or mechanical in nature, or a combination thereof. An example of a capture agent is avidin or streptavidin bound directly or indirectly (e.g., via a linker molecule) to the surface groups of glass, microparticles, plastic beads, gel beads, or the like.

Detergent: Any agent that is capble of emulsifying oil, and/or acts as a wetting agent or surfactant. Examples of useful detergents include but not limited to Triton X100, Tween 20, APO-10, APO-12, Big CHAP, Big CHAP, Deoxy, BRIJ® 35, PROTEIN GRADE® Detergent, 10% Solution, C₁₀E₆, C₁₀E8, C₁₂E₆, C₁₂E8, C₁₂E9, Cyclohexyl-n-ethyl-β-D-maltoside, Cyclohexyl-n-hexyl-β-D-maltoside, Cyclohexyl-n-methyl-β-D-maltoside, n-Decanoylsucrose, n-Decyl-β-D-glucopyranoside, n-Decyl-β-D-maltoside, n-Decyl-β-D-thiomaltoside, Digitonln, Digitonln, High Purity, n-Dodecanaylsucrose, n-Dodecyl-β-D-glucopyranoside, n-Dodecyl-β-D-maltoside, ELUGENT™ Detergent, GENAPOL® C-100, PROTEIN GRADE® Detergent GENAPOL® X-80, PROTEIN GRADE® Detergent, GENAPOL® X-100, PROTEIN GRADE® HECAMEG, n-Heptyl-β-D-glucopyranoside, n-Heptyl-β-D-thioglucopyranoside, 10% Solution, n-Hexyl-β-D-glucopyranoside, MEGA-8, MEGA-9, MEGA 10, n-Nonyl-β-D-glucopyranoside, NP-10, NP-40, PROTEIN GRADE® Detergent, n-Octanoyl-β-D-glucosylamine (NOGA), n-Octanoylsucrose, n-Octyl-β-D-glucopyranoside, n-Octyl-β-D-glucopyranoside, n-Octyl-β-D-maltopyranosiden-Octyl-β-D-thioglycopyranoside, PLURONIC® F-68, PLURONIC® F-127, PROTEIN GRADE®, n-Undecyl-β-D-maltoside and the like.

### 6. DETAILED DESCRIPTION OF THE PREFERRED EMBODIMENTS

The present invention contemplates an assay for the combined reactions of MraY and MurG. The invention describes attachment of a label, preferrably a biotin moiety, to the 3^{rd} amino acid of purified UDP-MurNAc pentapeptide (either a meso-DAP or Lys) using the epsilon amine. The biotinylated pentapeptide was then combined with either bacterial membranes (from normal bacteria or the ones overexpressing MraY or MurG) or a combination of membranes and purified enzymes. The mraY enzyme would attach the biotinylated pentapeptide to the undecaprenyl phosphate in the membrane, converting it to Lipid I. The MurG enzyme would convert the Lipid I to Lipid II in the presence of radiolabeled UDP-GlcNAc. The radiolabeled Lipid II was then captured by avidin attached to a solid phase. It may be necessary to disrupt the membrane with salt, detergent, pressure or abrasion prior to capture. Also, the non-specifically attached cell components must be washed away in the solid phase.

One of the differences, among others, that exists between this assay and the prior art assays is that in this assay a natural substrate, the UDP-murNAc pentapeptide, is labeled. UDP-MurNAc pentapeptide is naturally formed in the bacteria and thus can be isolated rather than synthesized. This intermediate is attached to a full length, natural lipid, e.g. undecaprenyl pyrophosphate, to form Lipid I. The labeled substrate is then incubated with membranes that is prepared, for example, by the French Press. The labeled substrate then is incorporated first into Lipid I, then into Lipid II and it can even be incorporated into peptidoglycan.

Furthermore, This invention discloses assay conditions that block incorporation of the labeled substrate into peptidoglycan, so one can measure the activities of both MraY and murG.

There are circumstances when it is desirable to measure the activities of trasglycosylase or translocase enzymes, in addition to Mra Y and MurG, in a broad screen assay. It was found, unexpectedly, that under certain assay conditions, whereby no detergent (i.e., Triton X) is added to the buffer, the labeled substrate is also incorporated into peptidoglycan. Therefore, this assay can be applied to measure transglycosylase and translocase activities as well as MraY and MurG activities. Thus, by allowing the UDP-MurNAc intermediate to continue on to polymerizing peptidoglycan, in the absence of Triton-X, the activities of known and unknown enzymes in E. coli could be measured. The assay conditions disclosed and described herewith produce a lower back ground and higher signal, so the assay can be run with lower radioactivity, thereby decreasing the cost.

The assay also uses membranes from bacteria that either overexpress or do not overexpress the MurG gene. The difference between the assay of this invention and the prior art assays lies in its broader scope and the fact that it is more reflective of the natural processes. This may be important for accurately predicting whether the assay inhibitors will function in a real cell environment.

Another main advantage of the assay of this invention is that it is more specific, since the end-product of the reaction is purified and is not contaminated by the cell components that incorporate radioactivity by other pathways.

This invention allows the measurement of the two major membrane-associated downstream enzymes in the peptidoglycan biosynthesis to be measured in tandem. So far no other assays has accomplished this. Another advantage of this assay is that it provides an improvement in the standard ether permeabilize cell assay for both the Gram negative and Gram positive bacteria for which no consistent assays exist. Up to now there has not been a reproducible assay for Gram positive bacteria and because of the genetic differences that exists between the enzymes of the Gram positive and Gram negative bacteria, the results obtained with Gram negative bacteria has not always been applicable to Gram positive bacteria. However, using the method described and disclosed herein made it possible to measure these as-yet-uncharacterized enzymes of Gram positive bacteria in membranes that do not overexpress the genes.

Both Mra Y and MurG enzymes could be recombinatly produced. The MurG gene product was cloned from E. coli, HIS-tagged for purification, and tested for its ability to complement the E. coli, MurG mutant strain OV58. Plasmids expressing the cloned enzyme (with or without a 5′-HIS-tag) were found to complement the mutation. The purification of the HIS-tagged enzyme using a nickel column resulted in an enzyme preparation that had concentration dependent activity in an E. coli peptidoglycan polymerization assay.

The MraY gene has also been cloned and manipulated by PCR for HIS-tagging and subsequent enzyme purification. The combined purified enzymes (MraY and MurG gene products), is used in the development of a sensitive high throughput assay to identify inhibitory compounds affecting the formation of peptidoglycan.

As a further illustration of the invention, the following examples are provided.

### 7. EXAMPLES

The following procedures are provided making specific reference to methods described in detail, below.

7.1 Isolation of Bacterial Membranes:

Bacterial membranes are used as a source of enzymes in the coupled MraY/MurG assay for the formation of Lipid II. Wild-type E. coli and MurG over-expressing constructs have been utilized as the membrane donors. Bacteria are grown in Brain Heart Infusion media supplemented with Casamino Acids (BHI/CAA) at 37° C. with aeration, to log phase (OD₆₀₀=0.7) and centrifuged at 7000×g. Pellets are washed once with 50× the wet pellet weight of 5 mM Tris-HCl (pH 8.0). Pellets are resuspended at a concentration of 2 gms wet pellet/35 mls 5 mM Tris for French Press disruption. Cells are pressure treated at 20,000 psi for 1 minute in a prechilled pressure cell. The membranes generated are collected by ultracentrifugation at 200,000×g for 1 hour at 4° C. Membrane pellets are resuspended in approximately the same volume as the wet pellet weight. This generally yields a membrane concentration of roughly 5 mg/ml. Aliquots are made, and membranes frozen at −80° C. until analysis.

7.2 Purification of UDP-MurNAc Pentapeptide from Bacteria:

The substrate for Lipid II formation is isolated using modifications of the procedure described by Kohlraush and Holtje, in FEMS Microbiol. Lett. (1991) 78:253-258. Assays are preferably performed using the L-lys form of UDP-MurNAc-pentapeptide isolated from the gram positive organism Enterococcus faecium. Bacteria are grown to log phase in BHI/CAA at 37° C. with aeration. Chloramphenicol is added to a final concentration of 170 ug/ml, and the culture is allowed to incubate for an additional 15 minutes. 10 ug/ml of Vancomycin is then added to the culture and incubation is allowed to proceed for an additional 60 minutes. This allows for the accumulation of the peptidoglycan precursor, UDP-MurNAc-pentapeptide. The culture is then chilled on ice, and the bacterial pellet is collected. The pellet is resuspended at a concentration of 0.1 gm/ml in dH₂O. The cells are slowly added to 2× volumes boiling water, and boiled for 15 minutes with stirring. The cells are slowly cooled to room temperature with constant stirring, and then chilled on ice. The resulting slurry is sonicated for a total of 1.3 minutes to completely disrupt the cells. The solution is ultracentrifuged as described above. The resulting supernatant is lyophilized to concentrate the sample. The lyophilisate is resuspended at a concentration of 5 mls dH₂O for each 10 liters-equivalent cells. 20% H₃PO₄ is added stepwise in 4 equal portions to lower the pH to 2.0. The solution is centrifuged at 12,000×g to remove precipitate after each addition of acid. The resulting solution is subjected to HPLC purification.

A Phenomenex semi-prep column is used to purify the UDP-MurNAc-pentapeptide from the other precursors. 50 mM sodium phosphate (pH 5.2) is used as the mobile phase under isocratic conditions. UV absorbance is monitored at 265 nm and is used to identify the UDP-MurNAc-pentapeptide as it elutes. Fractions are collected, pooled from the individual runs, and lyophilized to concentrate the samples. The samples are desalted by resuspending the lyophilisate in a minimal volume of dH₂O (approximately 5 mls) and rerunning the HPLC using different mobile phase conditions, so that the UDP-MurNAc-pentapeptide is eluted in 15% methanol (5 min Sodium Phosphate buffer, then shift to 15% methanol for 1 hour). The UDP-MurNAc pentapeptide peak is collected, its concentration is determined and aliquots are made and lyophilized. Samples are stored at −80° C. until labeling or assay development are performed.

7.3 Biotin-Labeling of UDP-MurNAcpentapeptide:

The purified UDP-MurNAc-pentapeptide containing a free amine is reacted with biotinamidocaproic acid 3-sulfo-N-hydroxy-succinimide ester in ratios of 1:2 equivalents respectively. The procedure is a modification of the method described elsewhere (see, Men, et. al., in J. Am. chem. Soc. (1998) 120:2484-2485). Two (2) equivalents of the biotin label plus one (1) equivalent of UDP-MurNAc pentapeptide, plus fifty (50) equivalents sodium bicarbonate are allowed to react at room temperature for 2 hours. The samples are speed vacuum dried, and purified by HPLC. Purification conditions involved using a Supercosil C-18 column (4.6 mm×25 cm) and a gradient mobile phase, initiating with 10 mM KPO₄ (pH 5.2) for 15 minutes, then switching to a water-methanol gradient, going from 0% MeOH to 50% MeOH over a period of 30 minutes. This not only separates biotin-labeled UDP-MurNAc pentapeptide from unlabeled, but also desalts the sample at the same time, minimizing the subsequent steps needed for purification. This allows for the small scale purification of labeled precursor. Conditions can be scaled up using semi-preparative HPLC columns. The labeled compound is lyophilized, resuspended in water to a concentration of approximately 1 nmole/10 ul, aliquoted and stored (and is completely stable) at −20° C. until use.

7.4 Lipid II Formation Assay:

Reactions for the formation of Lipid II have been optimized in 1.5 ml Eppendorf tubes. Reaction mixtures consist of the following reagents added individually, or as a master mix when identical reactions are being performed:

| Buffer formulation: | Assay Components (100 ul assay volume): |
|---|---|
| 50 mM Tris (pH = 8.0) | 50 ug French Pressed bacterial membranes |
| 42 mM MgAc | 0.5 uM ¹⁴C-GlcNAc |
| 208 mM KCl | 1-2 nmoles biotinylated |
| | UDP-MurNAc-pentapeptide |
| 0.1% Triton | |

The buffer is made as a 5× reagent, and is added to an appropriate volume of water to bring the final reaction volume to 50 ul. Biotin-labeled UDP-MurNAc-pentpeptide is added to the mixture followed by the addition of bacterial membranes (optimized for each membrane preparation (10-100 ug/reaction)). Reactions to test the effects of inhibitors involves the addition of the test compound at time zero. Reaction tubes are allowed to pre-incubate for 10 minutes before the introduction of radiolabeled ¹⁴C-GlcNAC. The introduction of the radiolabel signals the start of the incorporation reaction. This reaction is allowed to proceed for 15 minutes before being terminated by the addition of 25 ul of 1% SDS.

7.5 Streptavidin Capture

To the Eppendorf tubes containing the reaction mixtures, 500 ul of binding buffer (10 mM Tris-HCl (PH 8.0), 150 mM NaCl, 0.2% Triton X-100) is added. 25 ul of Tetralink (Promega) Tetrameric Avidin Resin is added to each reaction tube to allow for the streptavidin coated beads to capture the biotin-containing components. The tubes are gently mixed for 1 hour at room temperature. Samples are centrifuged for 3 minutes at 1500×g and resuspended in 500 ul of the binding buffer. The centrifugation and washing steps are repeated for a total of 4 times. The resulting beads are resuspended in buffer, mixed with scintillation cocktail, and counted in a scintillation counter to determine the amount of radioactivity associated with the capture event. The double labeling design of the assay allows only for the capture and detection of product that simultaneously contains the biotinylated UDP-MurNAc-pentapeptide and the ¹⁴C-labeled GlcNAc.

7.6 Paper Chromatography

Characterization of the assay necessitates the separation of potential products that incorporate ¹⁴C-GlcNAc. Whatmann 3MM chromatography paper is cut into 1 cm×20 cm strips. Ten ul of the reaction mixtures are dried onto a small spot on the paper, 1 cm from the bottom of the strip. The paper is placed into a large test tube containing 1 ml of isobutyric acid:1N NH₄OH (5:3 ratio). The solvent front is allowed to migrate to 1 cm from the top of the paper strip. The paper is then cut into 1 cm squares, and the individual strips are added to scintillation cocktail and counted. This technique gives supporting evidence that the biotin captured product is Lipid II.

7.7. ³H-NSP-Labeling of UDP-MurNAc-Pentapeptide

50 uCi (5×10⁻¹¹ moles, 1 mCi/mL, 100 Ci/mmol) n-Succinimidyl propionate is dried under N₂ and is added to 16.7 uL 10 uM UDP-MurNAc-pentapeptide in 0.1 M Borate buffer pH 3.3. (1:3.3 mole ratio NSP:UDP-pentapentide). The mixture is incubated for 30 min at 4° C. with agitation. Quench reaction is carried out with 16.7 uL 2 M glycine in borate buffer and is incubated 30 min at 4° C. with agitation. Purification is carried out on C18 HPLC using 50 mM sodium phosphate pH 5.2. Product is eluted at Rt˜15 minutes.

### 8. EQUIVALENTS

Those skilled in the art will recognize, or be able to ascertain using no more than routine experimentation, many equivalents to the specific embodiments of the invention described specifically herein. Such equivalents are intended to be encompassed in the scope of the following claims.

What is claimed is:

1. A uridine diphosphate N-acetylmuramyl peptide analog of the formula (UMP)-X, wherein the group UMP represents a uridine diphosphate N-acetylmuramyl peptide and the goup X represents a capture moiety that is directly or indirectly attached to said UMP and which capture moiety permits the separation of any substance to which said capture moiety is attached from a mixture comprising such substance.

2. The analog of claim 1 in which the peptide of said UMP comprises three or more amino acid residues.

3. The analog of claim 2 in which the group X is attached to one of the amino acid residues.

4. The analog of claim 3 in which the amino acid residue to which the group X is attached comprises a lysine residue.

5. The analog of claim 1 in which the peptide of said UMP comprises two or more amino acid residues and a meso-diaminopimelic acid (meso-DAP).

6. The analog of claim 5 in which the group X is attached to the meso-DAP.

7. The analog of claim 1 in which said capture moiety comprises a member of a pair of affinity binding substances.

8. The analog of claim 7 in which said member comprises biotin moiety.

9. The analog of claim 8 in which said biotin moiety is attached to the epsilon amine of a lysine residue forming part of said peptide.

10. A composition useful for the detection of bacterial cell wall biosynthesis enzyme activity in a test sample comprising an effective amount of a uridine diphosphate N-acetylmuramyl peptide analog of the formula (UMP)-X, wherein the group UMP represents a uridine diphosphate N-acetylmuramyl peptide and the goup X represents a capture moiety that is directly or indirectly attached to said UMP and which capture moiety permits the separation of any substance to which said capture moiety is attached from a mixture comprising such substance, said analog capable of serving as a substrate for at least one bacterial cell wall biosynthesis enzyme involved in the synthesis of Lipid I.

11. The composition of claim 10 which further comprises a labeled uridine diphosphate N-acetylglucosamine (UDP-GlcNAc), which is capable of serving as a substrate for at least one bacterial cell wall biosynthesis enzyme involved in the synthesis of Lipid II.

12. The composition of claim 11 in which said labeled UDP-GlcNAc bears a label selected from the group consisting of enzymes, biotin, fluorochromes, radioactive compounds, metals, chelators, peptides, nucleic acids, receptors and lectins.

13. The composition of claim 11 in which the group X is selected from the group consisting of peptides, proteins, nucleic acids and metal chelators.

14. The composition of claim 13 in which the group X comprises a member of a pair of affinity binding substances.

15. The composition of claim 14 in which said member comprises a receptor, a receptor ligand, biotin, or lectin.

16. A method of screening for potential antibacterial agents comprising:
(a) providing a bacterial membrane preparation or enriched enzyme preparation including at least one bacterial cell wall biosynthesis enzyme involved in the synthesis of Lipid I from UMP and undecaprenyl phosphate;
(b) providing an amount of an inhibitor effective to inhibit further processing of any Lipid I or Lipid I analog;
(c) providing an analog of UMP capable of serving as a substrate for the at least one bacterial cell wall biosynthesis enzyme involved in the synthesis of Lipid I, to provide a Lipid I analog;
(d) determining a baseline amount of Lipid I analog produced from the above-recited steps; and
(e) comparing such baseline amount with a test amount of Lipid I analog produced under the same conditions used to provide such baseline amount except for the presence of an added amount of a test agent suspected of exhibiting antibacterial activity.

17. The method of claim 16 in which analog of UMP comprises a capture moiety and, optionally, further comprising a radioactive isotope.

18. The method of claim 17 in which said capture moiety comprises one member of a pair of affinity binding substances.

19. The method of claim 16 in which said inhibitor comprises ramoplainin or tunicamycin.

20. A method of screening for potential antibacterial agents comprising:
(a) providing a bacterial membrane preparation or enriched enzyme preparation including at least one bacterial cell wall biosynthesis enzyme involved in the synthesis of Lipid I from UMP and undecaprenyl phosphate and at least one bacterial cell wall biosynthesis enzyme involved in the synthesis of Lipid II from Lipid I and UDP-GlcNAc;
(b) providing an amount of an inhibitor effective to inhibit further processing of any Lipid II or Lipid II analog;
(c) providing an analog of UMP capable of serving as a substrate for the at least one bacterial cell wall biosynthesis enzyme involved in the synthesis of Lipid I, to provide a Lipid I analog;
(d) providing a labeled UDP-GlcNAc capable of serving as a substrate for the at least one bacterial cell wall biosynthesis enzyme involved in the synthesis of Lipid II, to provide a labeled Lipid II analog;
(e) determining a baseline amount of labeled Lipid II analog produced from the above-recited steps; and
(f) comparing such baseline amount with a test amount of labeled Lipid II analog produced under the same conditions used to provide such baseline amount except for the presence of an added amount of a test agent suspected of exhibiting antibacterial activity.

21. The method of claim 20 in which said analog of UMP comprises UMP linked directly or indirectly to one member of a pair of affinity binding substances.

22. The method of claim 20 in which said labeled UDP-GlcNAc comprises UDP-GlcNAc bearing a radioactive isotope.

23. The method of claim 21 which further comprises contacting said labeled Lipid II analog with a capture agent.

24. The method of claim 23 in which said capture agent comprises a binding partner of the member of said pair of binding substances.

25. The method of claim 24 in which the binding partner is linked directly or indirectly to a solid substrate.

26. A method of screening for potential antibacterial agents comprising:
(a) providing a bacterial membrane preparation or enriched enzyme preparation including at least one bacterial cell wall biosynthesis enzyme involved in the synthesis of Lipid I from UMP and undecaprenyl phosphate, at least one bacterial cell wall biosynthesis enzyme involved in the synthesis of Lipid II from Lipid I and UDP-GlcNAc and one or more bacterial cell wall biosynthesis enzymes involved in the further processing of Lipid II toward the downstream synthesis of peptidoglycan;
(b) providing an analog of UMP capable of serving as a substrate for the at least one bacterial cell wall biosynthesis enzyme involved in the synthesis of Lipid I, to provide a Lipid I analog;
(c) providing a labeled UDP-GlcNAc capable of serving as a substrate for the at least one bacterial cell wall biosynthesis enzyme involved in the synthesis of Lipid II, to provide a labeled Lipid II analog;
(d) permitting the further processing of labeled Lipid II analog toward the downstream synthesis of peptidoglycan, to provide a peptidoglycan moiety that harbors the label of said labeled Lipid II analog;
(e) determining a baseline amount of said peptidoglycan moiety produced from the above-recited steps; and
(f) comparing such baseline amount with a test amount of peptidoglycan moiety produced under the same conditions used to provide such baseline amount except for the presence of an added amount of a test agent suspected of exhibiting antibacterial activity.

26. The method of claim 25 in which said analog of UMP comprises UMP linked directly or indirectly to one member of a pair of affinity binding substances.

27. The method of claim 25 in which said labeled UDP-GlcNAc comprises UDP-GlcNAc bearing a radioactive isotope.

28. The method of claim 26 which further comprises contacting said peptidoglycan moiety with a capture agent.

29. The method of claim 28 in which said capture agent comprises a binding partner of the member of a pair of binding substances.

30. The method of claim 29 in which said binding partner is linked directly or indirectly to a solid substrate.

31. The method of claim 25 in which the analog of UMP further comprises a label selected from the group consisting of a radioactive isotope, a chemiluminescent agent, or a fluorescent agent.

32. An assay kit comprising:
(a) an analog of UMP capable of serving as a substrate for at least one bacterial cell wall biosynthesis enzyme involved in the synthesis of Lipid I;
(b) a labeled UDP-GlcNAc capable of serving as a substrate for at least one bacterial cell wall biosynthesis enzyme involved in the synthesis of Lipid II; and
(c) a capture agent.

33. The assay kit of claim 32 in which said capture agent comprises a member of a pair of affinity binding substances, which member is bound directly or indirectly to a solid substrate.

34. The assay kit of claim 32 which further comprises a bacterial membrane preparation, an enriched enzyme preparation, or instructions for obtaining such preparations, including at least one bacterial cell wall biosynthesis enzyme involved in the synthesis of Lipid I from UMP and undecaprenyl phosphate.

35. The assay kit of claim 34 which further comprises an amount of an inhibitor effective to inhibit further processing of any Lipid I or Lipid I analog.

36. The assay kit of claim 32 which further comprises a bacterial membrane preparation, enriched enzyme preparation, or instructions for obtaining such preparations, including at least one bacterial cell wall biosynthesis enzyme involved in the synthesis of Lipid I from UMP and undecaprenyl phosphate and at least one bacterial cell wall biosynthesis enzyme involved in the synthesis of Lipid II from Lipid I and UDP-GlcNAc.

37. The assay kit of claim 36 which further comprises an amount of an inhibitor effective to inhibit further processing of any Lipid II or Lipid II analog.

38. The assay kti of claim 32 which futher comprises a bacterial membrane preparation, enriched enzyme preparation, or instructions for obtaining such preparations, including at least one bacterial cell wall biosynthesis enzyme involved in the synthesis of Lipid I from UMP and undecaprenyl phosphate, at least one bacterial cell wall biosynthesis enzyme involved in the synthesis of Lipid II from Lipid I and UDP-GlcNAc and one or more bacterial cell wall biosynthesis enzymes involved in the further processing of Lipid II toward the downstream synthesis of peptidoglycan.
